(19) United States
(12) Patent Application Publication
(10) Pub. No.: US 2009/0030321 A1
(43) Pub. Date: Jan. 29, 2009

(54) ULTRASONIC DIAGNOSTIC APPARATUS AND SOUND OUTPUT METHOD FOR ULTRASONIC DIAGNOSTIC APPARATUS

(76) Inventors: Tatsuro Baba, Otawara-shi (JP); Naohisa Kamiyama, Otawara-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/176,461

(22) Filed: Jul. 21, 2008

(30) Foreign Application Priority Data
Jul. 24, 2007 (JP) 2007-192256

(51) Int. Cl. A61B 8/06

(52) U.S. Cl. 600/454

(57) ABSTRACT

A vector norm N, an azimuthal angle θ and an angle of elevation φ which represent the velocity (blood flow velocity) of a specimen such as blood flow are acquired by a three-dimensional angle correction velocity vectorization section as three-dimensional fluid vector data indicating the three-dimensional flow direction and flow volume of the specimen such as the blood flow on the basis of Doppler signals corresponding to reception beams F₁ to F₄ received from a range gate RG by a two-dimensional ultrasonic probe. An audio output [y] for a three-dimensional sound system is generated by a velocity vector conversion processing section on the basis of the three-dimensional fluid vector data [N, θ, 100 ], and a three-dimensional sound system is driven.

### CROSS-REFERENCE TO RELATED APPLICATIONS

This application is based upon and claims the benefit of priority from prior Japanese Patent Application No. 2007-192256, filed Jul. 24, 2007, the entire contents of which are incorporated herein by reference.

### BACKGROUND OF THE INVENTION

1. Field of the Invention

The present invention relates to an ultrasonic diagnostic apparatus for performing the conversion into sound and the output of, for example, the flow direction of a specimen which is a fluid such as blood flowing in a living body such as a human body, and the present invention also relates to a sound output method thereof.

2. Description of the Related Art

Ultrasonic Doppler diagnostic apparatuses are classified into pulse wave Doppler (PWD) and continuous wave Doppler (CWD) depending on the kind of ultrasonic beam sent from an ultrasonic probe. In both the pulse wave Doppler (PWD) and the continuous wave Doppler (CWD), the ultrasonic Doppler diagnostic apparatus utilizes a Doppler effect wherein when an ultrasonic wave is reflected by, for example, a moving blood flow and tissue in a human body, the frequency of a wave reflected by the blood flow and tissue is slightly different from the frequency of an incident wave. Then, the ultrasonic Doppler diagnostic apparatus uses the Doppler effect to measure the velocity of the blood flow and tissue in, for example, a human body or two-dimensionally displays the blood flow in color.

In order to tell a Doppler signal output from the ultrasonic probe, there are, for example, a method to analyze the frequency of the Doppler signal to convert it into a Doppler frequency corresponding to the velocity and display the Doppler frequency, and a method to output the Doppler signal directly through speakers with sound. Of these methods, in the method to output with sound, the Doppler signals of the pulse wave Doppler (PWD) and the continuous wave Doppler (CWD) are separated by direction, that is, the blood flows are separated in accordance with the directions, and the blood flow moving toward the ultrasonic probe is defined as positive while the blood flow moving away from the ultrasonic probe is defined as negative, and then these blood flows are output as audio from, for example, two speakers arranged on the right and left.

A user listens to Doppler sound output from the speakers arranged on the right and left, and in accordance with the presence of the Doppler sound, detects, with high sensitivity and high response, the presence of blood flowing in a small blood vessel within, for example, the liver in an ultrasonic tomogram of, for example, a human body. Then, the user determines a color region of interest (ROI) depending on the presence of blood flowing in the blood vessel. The user causes an ultrasonic beam to be applied by the pulse wave Doppler (PWD) to determine a range gate (RG) as a part where, for example, blood flow is to be measured.

However, there is a demand that the direction of blood flow be recognized in a three-dimensional space in a coordinate system around, for example, the center of the ultrasonic probe or around the range gate (RG) of a living body instead of separating the Doppler signals by direction and outputting the Doppler signals as audio from the two speakers arranged on the right and left.

It is an object of the present invention to provide an ultrasonic diagnostic apparatus and a sound output method for the ultrasonic diagnostic apparatus capable of acquiring the direction of blood flow in a three-dimensional space in a coordinate system around the center of an ultrasonic probe or around a range gate (RG) of a living body.

### BRIEF SUMMARY OF THE INVENTION

An ultrasonic diagnostic apparatus according to a first aspect of the present invention comprises: a three-dimensional information acquisition section which acquires three-dimensional fluid information including at least a three-dimensional flow direction of a specimen on the basis of a Doppler signal output from an ultrasonic probe, the specimen being at least a fluid in a particular part; and a three-dimensional sound output section which outputs the three-dimensional fluid information as a sound in a three-dimensional space.

A sound output method for an ultrasonic diagnostic apparatus according to a second aspect of the present invention comprises: acquiring three-dimensional fluid information including at least a three-dimensional flow direction of a specimen on the basis of a Doppler signal output from an ultrasonic probe, the specimen being at least a fluid in a particular part; and outputting the three-dimensional fluid information as a three-dimensional sound.

### BRIEF DESCRIPTION OF THE SEVERAL VIEWS OF THE DRAWING

FIG. 1 is a block configuration diagram of an ultrasonic Doppler diagnostic apparatus according to the present invention;

FIG. 2 is a schematic diagram of a two-dimensional probe surface of an ultrasonic probe in the same apparatus;

FIG. 3 is a diagram for explaining a Doppler angle correcting method applied to the same apparatus;

FIG. 4 is a specific configuration diagram showing one embodiment of the ultrasonic Doppler diagnostic apparatus according to the present invention;

FIG. 5 is a schematic diagram for explaining a Doppler angle correction in the same apparatus in a two-dimensional section;

FIG. 6 is a schematic diagram for explaining the Doppler angle correcting method applied to the same apparatus;

FIG. 7 is a schematic diagram for explaining the Doppler angle correcting method applied to the same apparatus;

FIG. 8 is a schematic diagram for explaining the Doppler angle correcting method applied to the same apparatus;

FIG. 9 is a diagram showing one example of the arrangement of a plurality of speakers in the same apparatus;

FIG. 10 is a diagram showing one example of frequency modulation corresponding to a Doppler signal in the same apparatus;

FIG. 11 is a diagram showing one example of amplitude modulation of a sinusoidal wave in accordance with a Doppler signal in the same apparatus;

FIG. 12 is a diagram showing one example of amplitude modulation using white noise in accordance with a Doppler signal in the same apparatus;

FIG. 13 is a diagram showing one example of a method of controlling a sound pressure with which the speakers arranged on the right and left with respect to an azimuthal angle in the same apparatus are driven; and

FIG. 14 is a diagram showing one example of a method of controlling a sound pressure with which the speakers for frequencies using an angle of elevation in the same apparatus as a parameter are driven.

### DETAILED DESCRIPTION OF THE INVENTION

Hereinafter, one embodiment of the present invention will be described with reference to the drawings.

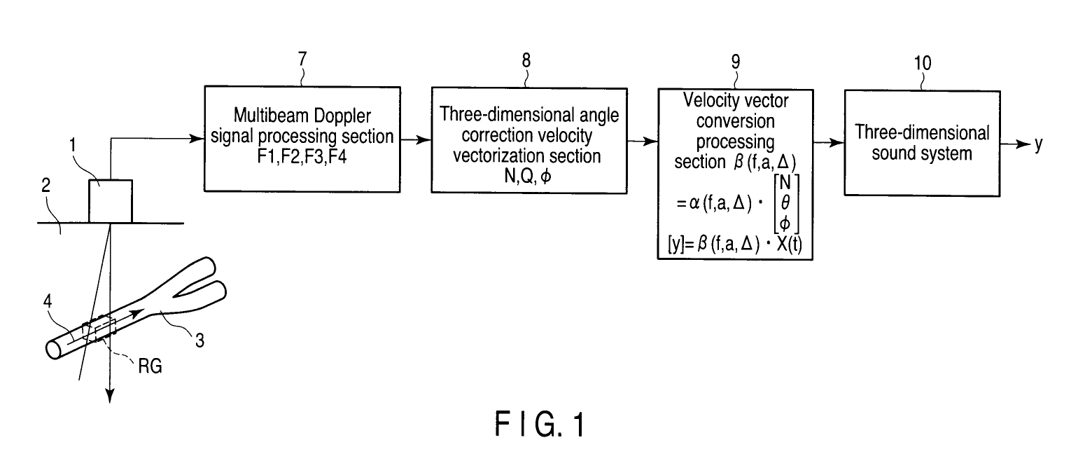

FIG. 1 shows a block configuration diagram of an ultrasonic Doppler diagnostic apparatus. A two-dimensional ultrasonic probe 1 sends an ultrasonic multibeam M composed of a plurality of beams to a particular region (hereinafter referred to as a range gate: RG) within a living body 2 such as a human body, and receives a reflected wave from the range gate RG. The range gate RG includes a specimen 4 which is a fluid such as blood flowing in a blood vessel 3 within the living body 2 such as the human body. The ultrasonic probe 1 comprises a plurality of ultrasonic transducers arranged on a two-dimensional plane. The ultrasonic probe 1 uses the plurality of ultrasonic transducers to send the ultrasonic multibeam M and receive the reflected wave.

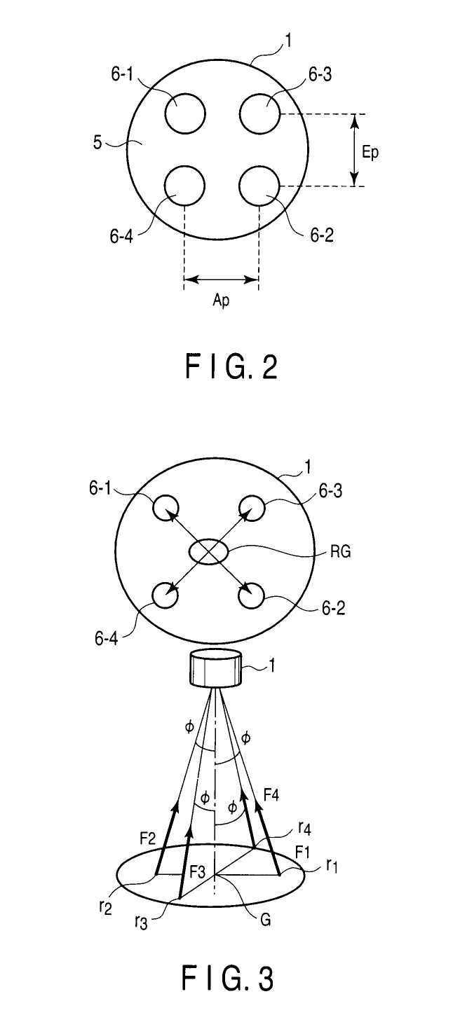

FIG. 2 schematically shows a two-dimensional probe surface of the ultrasonic probe 1. The ultrasonic probe 1 can receive the reflected wave from the range gate RG by, for example, ultrasonic transducers 6-1 to 6-4 at four places among a plurality of ultrasonic transducers arranged on the two-dimensional plane. In addition, when the ultrasonic transducers 6-1, 6-3, 6-4 are used, the distance between the ultrasonic transducer 6-1 and the ultrasonic transducer 6-4 is an elevation pitch Ep. The distance between the ultrasonic transducer 6-1 and the ultrasonic transducer 6-3 is an azimuth pitch Ap. Thus, the ultrasonic probe 1 receives reception beams F₁ to F₄ from the range gate RG by the ultrasonic transducers 6-1 to 6-4 at four places, as shown in, for example, FIG. 3.

A multibeam Doppler signal processing section 7 electronically scans the plurality of ultrasonic transducers of the ultrasonic probe 1. The multibeam Doppler signal processing section 7 detects Doppler signals from output signals of the ultrasonic transducers 6-1 to 6-4 which have received the reception beams F_{1 to F}₄ from the range gate RG.

A three-dimensional angle correction velocity vectorization section 8 acquires three-dimensional fluid vector data indicating a three-dimensional flow direction and volume of the specimen 4 such as blood flow on the basis of the Doppler signals of the ultrasonic transducers 6-1 to 6-4 detected by the multibeam Doppler signal processing section 7. The three-dimensional fluid vector data includes a vector norm N, an azimuthal angle θ and an angle of elevation φ which represent the velocity (blood flow velocity) of the specimen 4 such as blood flow.

The three-dimensional fluid vector data is represented by [N, θ, φ]. In addition, the three-dimensional angle correction velocity vectorization section 8 uses Doppler angle correction to calculate the norm N (blood flow velocity), etc., of the three-dimensional fluid vector data indicating the volume of the specimen 4 such as blood flow. The Doppler angle correction comprises measuring an angle between the direction of the ultrasonic beam and the flow direction (hereinafter referred to as the blood flow direction) of the specimen 4 such as blood flow, that is, a Doppler angle to find the absolute value of the blood flow velocity.

A velocity vector conversion processing section 9 converts the data into an audio output for a three-dimensional sound system 10 on the basis of the vector norm N, the azimuthal angle θ and the angle of elevation φ acquired by the three-dimensional angle correction velocity vectorization section 8. The velocity vector conversion processing section 9 allocates the three-dimensional fluid vector data [N, θ, φ] to artificial sound parameters by α (f, a, Δ) and thus converts the data to β (f, a, Δ). In addition, f is a frequency characteristic, a is an amplitude characteristic, and Δis the difference (a phase difference) between right and left paths of a binaural system.

$$β ( f , a , Δ ) = α ( f , a , Δ ) \cdot [ \begin{matrix}N \\ θ \\ φ\end{matrix} ]$$

The velocity vector conversion processing section 9 multiplies, by a three-dimensional sound space conversion β, a sound source X(t) generated on the basis of amplitude and frequency components from the Doppler signals detected from the reception beams F₁ to F₄ from the range gate RG, thereby generating an audio output [y] for a three-dimensional sound system.

[y]=β(f, a, Δ)·X(t)

The three-dimensional sound system 10 carries out the electro-acoustic transduction of the audio output [y] for the three-dimensional sound system generated by the velocity vector conversion processing section 9. The three-dimensional sound system is, for example, a multispeaker system, a sound scheme using 7.1 channels (7.1 surround-sound system), or a binaural system.

In such an ultrasonic Doppler diagnostic apparatus, the two-dimensional ultrasonic probe 1 sends the ultrasonic multibeam M composed of a plurality of beams to the range gate within the living body 2 such as a human body, and receives a reflected wave from the range gate RG. For example, as shown in FIG. 3, the ultrasonic probe 1 receives the reception beams F₁ to F₄ from the range gate RG by the ultrasonic transducers 6-1 to 6-4 at four places.

The multibeam Doppler signal processing section 7 electronically scans the plurality of ultrasonic transducers of the ultrasonic probe 1. The multibeam Doppler signal processing section 7 then detects Doppler signals from output signals of the ultrasonic transducers 6-1 to 6-4 which have received the reception beams F₁ to F₄ from the range gate RG.

The three-dimensional angle correction velocity vectorization section 8 acquires the vector norm N, the azimuthal angle θ and the angle of elevation φ which represent the velocity (blood flow velocity) of the specimen 4 such as blood flow, as the three-dimensional fluid vector data indicating the three-dimensional flow direction and volume of the specimen 4 such as blood flow on the basis of the Doppler signals of the ultrasonic transducers 6-1 to 6-4 detected by the multibeam Doppler signal processing section 7.

The velocity vector conversion processing section 9 allocates the three-dimensional fluid vector data [N, θ, φ] acquired by the three-dimensional angle correction velocity vectorization section 8 to the artificial sound parameters by α (f, a, Δ) and thus converts the data into P (f, a, Δ). The velocity vector conversion processing section 9 then multiplies, by the three-dimensional sound space conversion β, the sound source X(t) generated on the basis of the amplitude and frequency components from the Doppler signals, thereby generating the audio output [y] for the three-dimensional sound system.

The three-dimensional sound system 10 drives, for example, the multispeaker system, the sound scheme using 7.1 channels (7.1 surround-sound system), or the binaural system to carry out the electro-acoustic transduction of the audio output [y] for the three-dimensional sound system generated by the velocity vector conversion processing section 9.

As described above, according to the one embodiment of the present invention, the multibeam Doppler signal processing section 7 detects the Doppler signals corresponding to the reception beams F₁ to F₄ received from the range gate RG by the two-dimensional ultrasonic probe 1. On the basis of the Doppler signals, the three-dimensional angle correction velocity vectorization section 8 acquires the vector norm N, the azimuthal angle θ and the angle of elevation φ which represent the velocity (blood flow velocity) of the specimen 4 such as blood flow, as the three-dimensional fluid vector data indicating the three-dimensional flow direction and volume of the specimen 4 such as blood flow. On the basis of the three-dimensional fluid vector data [N, θ, φ], the velocity vector conversion processing section 9 generates the audio output [y] for the three-dimensional sound system, and drives the three-dimensional sound system 10.

Thus, the direction of blood flow can be acquired in a three-dimensional space in a coordinate system around the center of the ultrasonic probe 1 or around the range gate (RG) of a living body. For example, an operator such as a doctor listens to a sound at a frequency corresponding to the velocity of blood flow, with a sound pressure corresponding to the azimuthal angle θ and with a frequency characteristic corresponding to the angle of elevation φ, and can aurally know the flow direction, volume, etc., of the specimen 4 which is a fluid such as blood flowing in the blood vessel 3.

Next, a specific example of the one embodiment of the present invention is described.

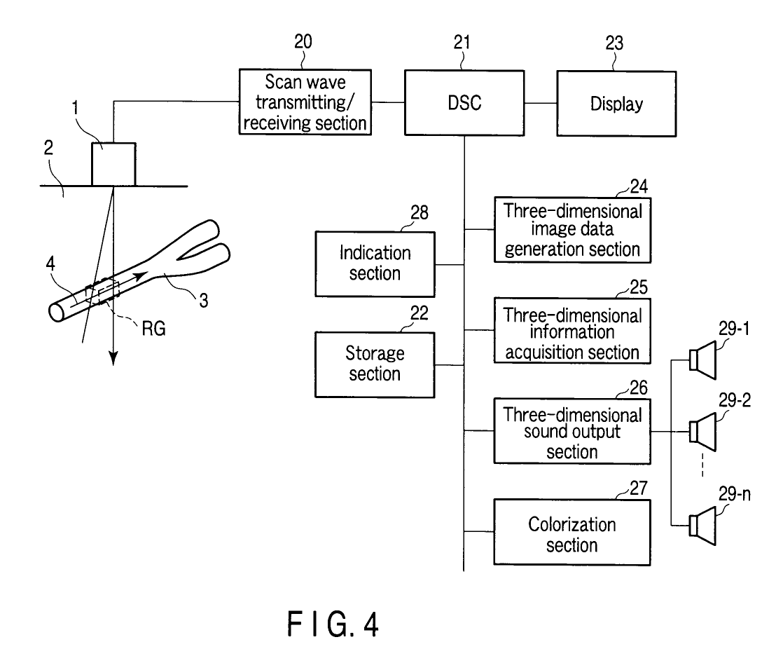

FIG. 4 shows a configuration diagram of the ultrasonic Doppler diagnostic apparatus. As in the case described above, the two-dimensional ultrasonic probe 1 sends the ultrasonic multibeam M composed of a plurality of beams to the range gate RG within the living body 2 such as a human body, and receives a reflected wave from the range gate RG. The ultrasonic probe 1 comprises a plurality of ultrasonic transducers arranged on a two-dimensional plane. The ultrasonic probe 1 uses the plurality of ultrasonic transducers to send the ultrasonic multibeam M and receive the reflected wave. As shown in FIG. 2, the ultrasonic probe 1 can receive the reflected wave from the range gate RG by, for example, the ultrasonic transducers 6-1 to 6-4 at four places among the plurality of ultrasonic transducers arranged on the two-dimensional plane.

A scan wave transmitting/receiving section 20 corresponds to the multibeam Doppler signal processing section 7 described above. The scan wave transmitting/receiving section 20, for example, electronically scans the plurality of ultrasonic transducers of the ultrasonic probe 1, and sequentially drives the ultrasonic transducers to scan with the ultrasonic multibeam M. Then, the scan wave transmitting/receiving section 20 detects Doppler signals from output signals of the ultrasonic transducers of the ultrasonic probe 1 when the reflected wave from, for example, the range gate RG is received.

A digital scan converter (hereinafter referred to as a DSC) 21 subjects the Doppler signals output from the scan wave transmitting/receiving section 20 to digital conversion, and then stores the signals in a storage section 22 such as an image memory. The DSC 21 reads the digital Doppler signals stored in the storage section 22 in accordance with the scanning of a display 23. The DSC 21 performs the analog conversion of the digital Doppler signals to display, on a display 23, an ultrasonic image of the range gate RG within the living body 2 such as a human body in real time. The DSC 21 has a three-dimensional image data generation section 24, a three-dimensional information acquisition section 25, a three-dimensional sound output section 26, a colorization section 27 and an indication section 28. The display 23 is connected to the DSC 21.

The three-dimensional image data generation section 24 performs the digital conversion of the Doppler signals output from the scan wave transmitting/receiving section 20, and stores, for example, digital Doppler signals for a preset scan period in the storage section 22, thereby acquiring a plurality of tomographic acquisition data (stack data). Then, the three-dimensional image data generation section 24 reconstructs the plurality of tomographic acquisition data (stack data), and generates three-dimensional ultrasonic image data (volume data) for the range gate RG within the living body 2 such as a human body.

The three-dimensional information acquisition section 25 corresponds to the three-dimensional angle correction velocity vectorization section 8. The three-dimensional information acquisition section 25 acquires the velocity (blood flow velocity) of the specimen 4 such as blood flow, that is, the vector norm N, the azimuthal angle 0 and the angle of elevation φ, as the three-dimensional fluid information including at least a three-dimensional flow direction of the specimen 4 in a particular part within the three-dimensional ultrasonic image data generated by the three-dimensional image data generation section 24, that is, in the range gate RG within the living body 2 such as a human body. The three-dimensional information acquisition section 25 acquires the vector norm N, the azimuthal angle θ and the angle of elevation φ on the basis of the fluid vector data indicating the three-dimensional flow direction and volume of the specimen 4 such as blood flow within the three-dimensional ultrasonic image data. Moreover, the three-dimensional information acquisition section 25 acquires the degree of turbulence and pulsation of the specimen 4 such as blood flow as the three-dimensional fluid information.

Here, the calculation of the norm N, etc., of the fluid vector data indicating the volume of the specimen 4 such as blood flow is described.

In an ultrasonic Doppler method, the angle between the direction of the ultrasonic beam and the blood flow direction of the specimen 4 is called the Doppler angle. In the measurement of the blood flow velocity by the ultrasonic Doppler method, a detected Doppler shift frequency is in proportion to the product of the cosines of the blood flow velocity and the Doppler angle, and dependent on the Doppler angle. Moreover, the measurement of the Doppler angle to find the absolute value of the blood flow velocity is called the Doppler angle correction. Thus, the Doppler angle correction is used to calculate the norm (blood flow velocity), etc., of the fluid vector data indicating the volume of the specimen 4 such as blood flow.

Now, the Doppler angle correction is described.

As shown in FIG. 3, all the angles of four directions of the elevation (angle of elevation) and the azimuth (azimuthal angle) across the range gate RG including the specimen 4 such as blood flow are equal to an angle φ. Further, the range gate RG including the specimen 4 such as blood flow is in the middle of four ultrasonic beams. There is a uniform blood flow in the range gate RG.

The angles (hereinafter referred to as angle of elevations) φ of the four directions of the elevation and the azimuth are small. Thus, the distances from a center G to reflection points r₁ to r₄ of the reception beams F₁ to F₄ are equal owing to a swing angle at which scanning is performed with the ultrasonic beams. The elevation angles φ are previously known.

Furthermore, the directions of the reception beams F₁ to F₄ are the same even in the center of the range gate RG. The reception beams F₁ to F 4 are indicated by vectors.

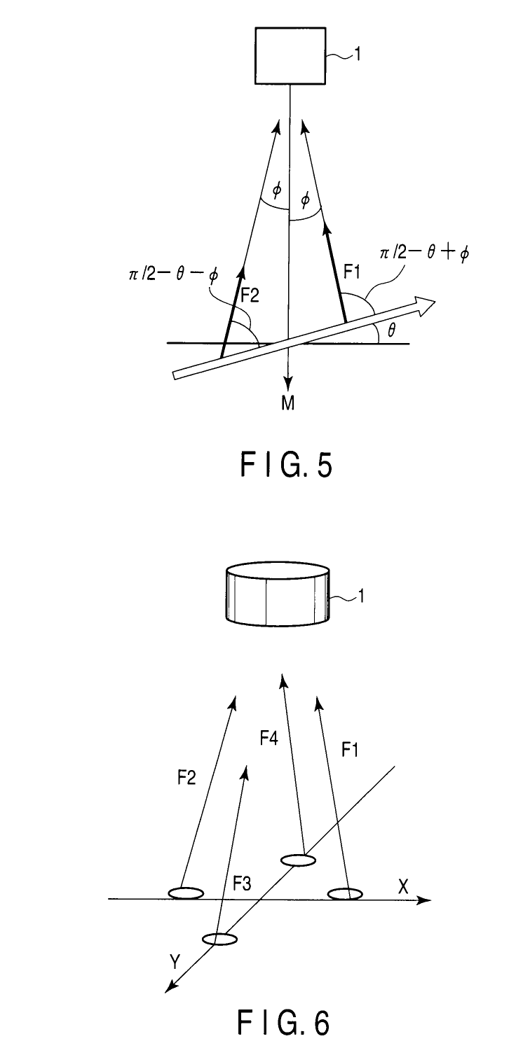

First, a calculation method in a two-dimensional section is described with reference to FIG. 5.

The reception beams F₁ to F₄ are received by the ultrasonic transducers 6-1 to 6-4 of the ultrasonic probe 1 at four places. The scan wave transmitting/receiving section 20, for example, electronically scans the plurality of ultrasonic transducers of the ultrasonic probe 1, and detects Doppler signals from output signals of the ultrasonic transducers 6-1 to 6-4. On the basis of the Doppler signals received by the ultrasonic transducers 6-1 to 6-4, the three-dimensional information acquisition section 25 performs the following calculations:

f₁=f₀*sin(π/2−θ+φ)

f₂=f₀*sin(π/2−θ−φ)

In other words,

f₁=f0*cos(θ−φ)

f₂=f₀*cos (θ+φ).

where, the scalar quantities of the reception beams F₁ to F₄ are f₁ to f₄. A fluid vector indicating the volume of the specimen 4 such as blood flow, that is, an unknown blood flow vector is F₀. f₀ indicates the blood flow velocity which is the scalar quantity of the blood flow vector F₀, that is, the vector norm N. Further, an angle θ is an azimuthal angle.

If the above equations are expanded,

f₁=f₀*(sin θ*cos φ−cos θ*sinφ)

f₂=f₀*(sin θ*cos φ+cos θ*sinφ)

Thus,

tan θ={(f₁+f₂)/(f₂−f₁)}*tan φ,

so that the azimuthal angle θ is found by the following equation:

θ=tan⁻¹[{(f₁+f₂)/(f₂−f₁)}*tan φ].

Moreover, the velocity f₀ of the specimen 4 such as blood flow after angle correction is found by the following equation:

$$f 0 = \frac{1}{2} * \sqrt{\frac{{( f 2 + f 1 )}^{2}}{{cos}^{2} φ} + \frac{{( f 2 - f 1 )}^{2}}{{sin}^{2} φ}}$$

If this equation is three-dimensionally expanded,

$$θ a = \frac{1}{2} \sqrt{\frac{{( f 2 + f 1 )}^{2}}{{cos}^{2} φ} + \frac{{( f 2 - f 1 )}^{2}}{{sin}^{2} φ}}$$

$$θ a = {tan}^{- 1} ( \frac{f 1 + f 2}{f 2 - f 1} * tan φ )$$

$$fe = \frac{1}{2} \sqrt{\frac{{( f 4 + f 3 )}^{2}}{{cos}^{2} φ} + \frac{{( f 4 - f 3 )}^{2}}{{sin}^{2} φ}}$$

$$θ e = {tan}^{- 1} ( \frac{f 4 + f 3}{f 4 - f 3} * tan φ )$$

is found.

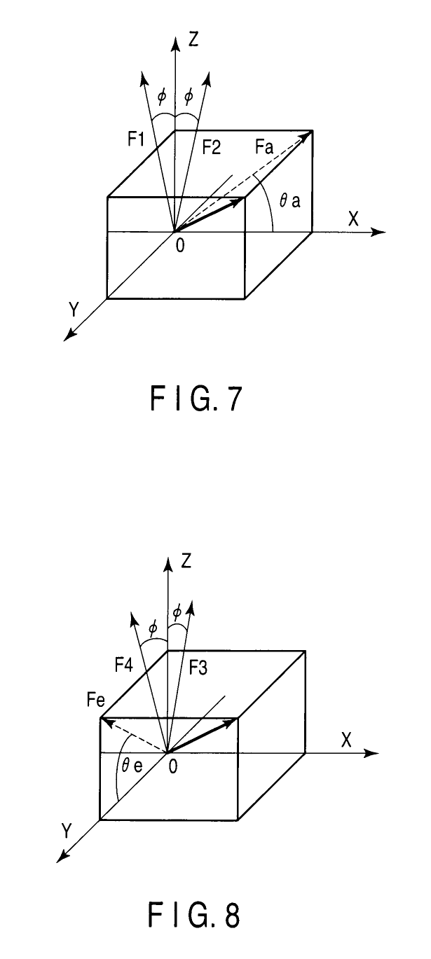

That is, as shown in FIG. 6 and FIG. 7, projection vectors of a section (X-Z plane) in an azimuth direction from each of the reception beams F₁, F₂ and a section (Y-Z plane) in an elevation direction from each of the reception beams F₃, F₄ are calculated using a two-dimensional technique.

As a result, the flow velocity f₀ of the three-dimensional blood flow vector F₀ is found.

\vec{f0}=(fa*cos θa, fe*cos θe, fe*sin θe) or (fa*cos θa, fe*cos θe, fa*sin θa)

|f0|=\sqrt{fe²+(fa*cos θa)²} or \sqrt{fa²+(fe*cos θe)²}

Thus, as the three-dimensional fluid information, the three-dimensional information acquisition section 25 acquires the velocity f₀ of the specimen 4 such as blood flow indicated by the three-dimensional blood flow vector F₀ originating from the range gate RG, that is, acquires the vector norm N, the azimuthal angle θ and the elevation angle φ. The elevation angle φ is previously known.

The three-dimensional sound output section 26 corresponds to the velocity vector conversion processing section 9. The three-dimensional sound output section 26 receives the vector norm N, the azimuthal angle θ and the elevation angle φ of the specimen 4 such as blood flow as the three-dimensional fluid information in the range gate RG acquired by the three-dimensional information acquisition section 25. The three-dimensional sound output section 26 performs the sound conversion of the Doppler signals from the scan wave transmitting/receiving section 20 into Doppler sounds in a three-dimensional space in accordance with the vector norm N, the azimuthal angle θ and the elevation angle φ, and outputs the Doppler sounds. A plurality of speakers 29-1 to 29-n are connected to the three-dimensional sound output section 26. These speakers 29-1 to 29-n are arranged in, for example, a two-dimensional or three-dimensional space.

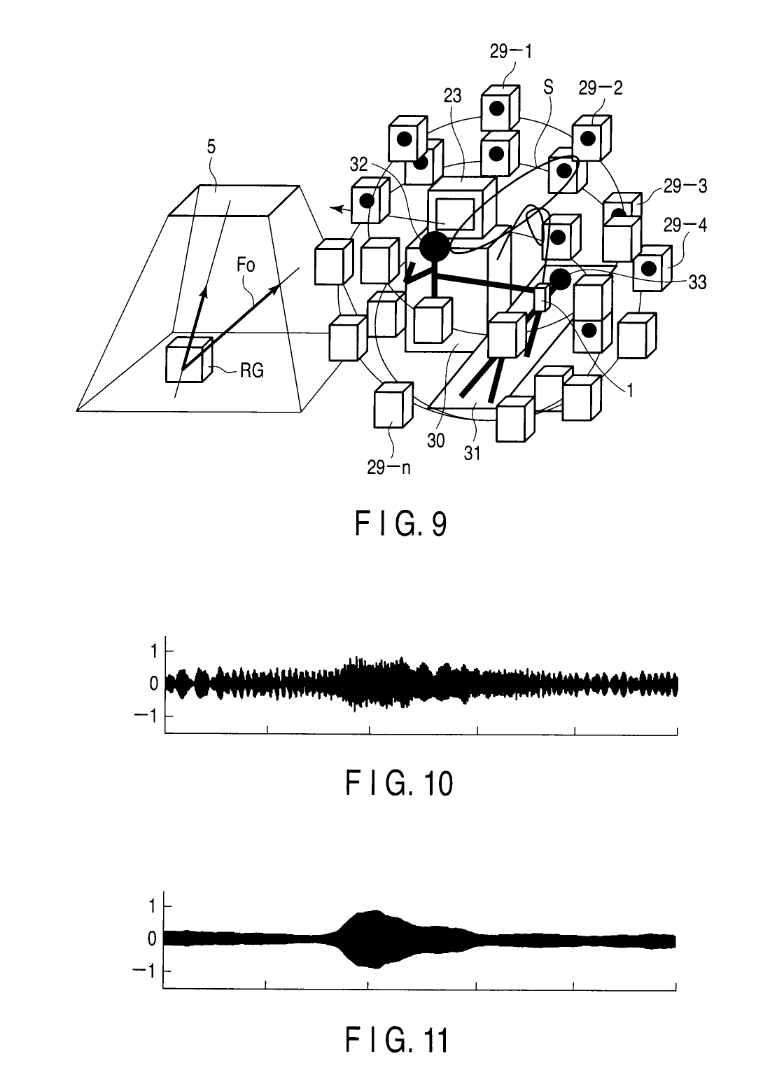

FIG. 9 shows one example of the arrangement of the speakers 29-1 to 29 -n. An ultrasonic Doppler diagnostic apparatus main body 30 is disposed. A bed 31 is disposed adjacently to the ultrasonic Doppler diagnostic apparatus main body 30. An operator 32 such as a doctor is present on the front side of the ultrasonic Doppler diagnostic apparatus main body 30. A subject 33 such as a patient is mounted on the bed 31. The operator 32 such as the doctor puts the ultrasonic probe 1 on the subject 33.

The plurality of speakers 29-1 to 29-n are arranged in the three-dimensional space surrounding the ultrasonic Doppler diagnostic apparatus main body 30, the operator 32, the bed 31, and the subject 33 on the bed 31. The plurality of speakers 29-1 to 29-n are arranged at even intervals on a plurality of circumferences around the position where the operator 32 such as the doctor is seated. The speakers 29-1 to 29-n are arranged on a spherical surface around the position where the operator 32 is seated. The circumferences on which the speakers 29-1 to 29-n are arranged are different in, for example, radius or position. The number of speakers 29-1 to 29-n is, for example, 60.

The three-dimensional sound output section 26 drives at least one of the speakers 29-1, 29-2, . . . , 29-n on the basis of the velocity f₀, the azimuthal angle θ and the elevation angle φ of the specimen 4 such as blood flow as the three-dimensional fluid information. Thus, the operator 32 such as the doctor listens to the sound of at least one of the speakers 29-1, 29-2, . . . , 29-n, and thereby listens to a sound S corresponding to the velocity f₀, the azimuthal angle θ and the elevation angle φ of the specimen 4 such as blood flow indicated by the three-dimensional blood flow vector F₀ originating from the range gate RG.

In this case, the three-dimensional sound output section 26 can make variations by combining at least one or two of the sound pressure, phase difference and frequency characteristic of the three-dimensional Doppler sound with which at least one of the speakers 29-1, 29-2, . . . , 29-n is driven in accordance with the velocity f₀, the azimuthal angle θ and the elevation angle φ of the specimen θ such as blood flow.

For example, the three-dimensional sound output section 26 changes at least one of the frequency characteristic, sound pressure and reverberation amount of the three-dimensional Doppler sound in accordance with the velocity f₀ of the specimen 4 such as blood flow, and then drives at least one of the speakers 29-1, 29-2, . . . , 29-n.

Furthermore, the three-dimensional sound output section 26 changes at least one of the sound pressure and phase difference of the three-dimensional Doppler sound in accordance with the azimuthal angle θ, and then drives at least one of the speakers 29-1, 29-2, . . . , 29-n. In this case, when the phase difference is varied in accordance with the azimuthal angle θ, the three-dimensional sound output section 26 provides the phase difference between two speakers, for example, speakers 16-1, 16-n, and then produces the Doppler sound.

Here, the following first to third sound schemes are available to drive at least one of the speakers 29-1, 29-2, . . . , 29-n in accordance with the velocity f₀, the azimuthal angle θ and the elevation angle φ of the specimen 4 such as blood flow.

In the first sound scheme, the frequency is varied when at least one of the speakers 29-1, 29-2, . . . , 29-n is driven by the three-dimensional sound output section 26 in accordance with the velocity f₀ of the specimen 4 such as blood flow. That is, for example, the three-dimensional sound output section 26 increases the frequency when the blood flow velocity f₀ is high, and decreases the frequency when the blood flow velocity f₀ is low.

There are, for example, two methods of varying the frequency in accordance with the blood flow velocity f₀.

In the first method, a maximum flow velocity Vp or average flow velocity Vm of the blood flow velocity f₀ of a spectrum is subjected to frequency modulation (FM). This frequency modulation allows the blood flow velocity f₀ to correspond to the frequency. Further, the total power of the spectrum is converted to create an envelope, and then amplitude modulation (AM) is performed.

In the second method, the three-dimensional sound output section 26 uses a sinusoidal wave, for example, at a frequency of 400 Hz or white noise as a sound source, and subjects the sinusoidal wave or white noise to the amplitude modulation in accordance with the velocity f₀ of the specimen 4 such as blood flow.

For example, as the sound source, the three-dimensional sound output section 26 uses noise which has been subjected to the amplitude modulation (AM) by the envelope of the waveform of the Doppler signal, such as the white noise.

Furthermore, the three-dimensional sound output section 26 filters, as the sound source, the sinusoidal wave or white noise in accordance with the center frequency and dispersion of the waveform of the Doppler signal, and subjects the signal generated by the filtering to the amplitude modulation (AM) by the envelope of the waveform of the Doppler signal, and then uses the modulated signal.

In addition, the three-dimensional sound output section 26 may carry out the frequency modulation instead of the amplitude modulation.

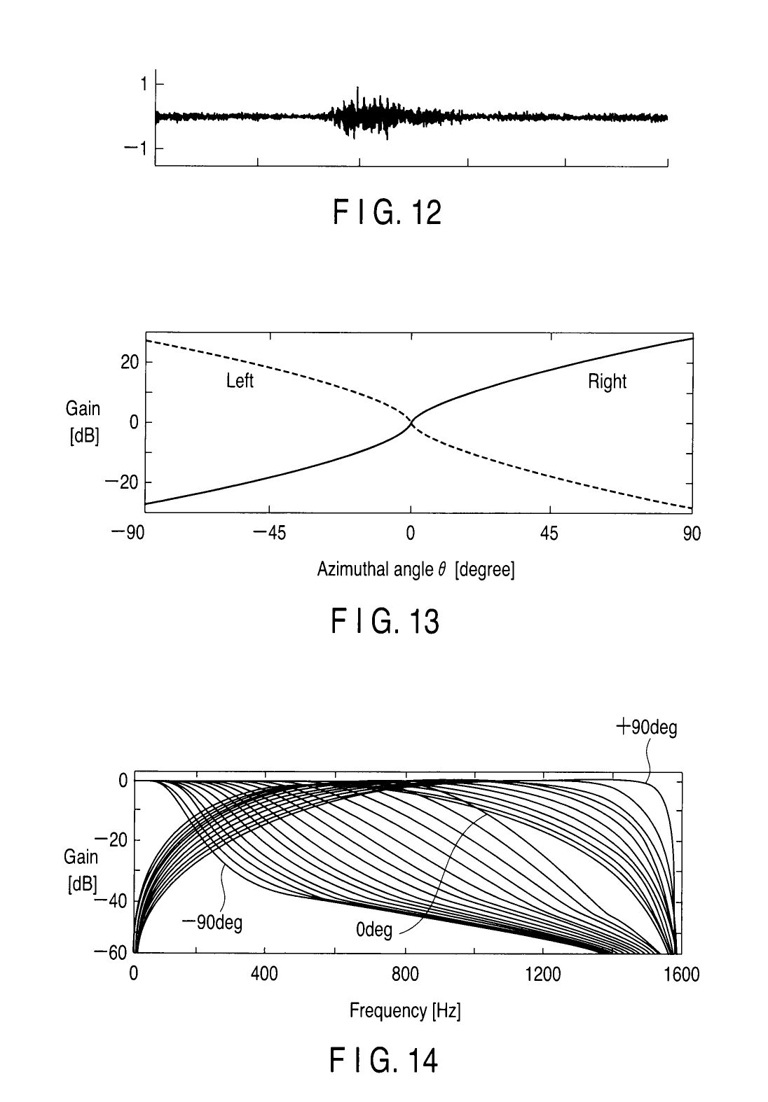

FIG. 10 shows one example of the frequency modulation corresponding to the Doppler signal. FIG. 11 shows one example of the amplitude modulation of a sinusoidal wave in accordance with the Doppler signal. FIG. 12 shows one example of the amplitude modulation using the white noise in accordance with the Doppler signal.

In connection with this, the three-dimensional sound output section 26 varies the sound pressure with which at least one of the speakers 29-1, 29-2, . . . , 29-n is driven in accordance with the azimuthal angle θ. For example, the three-dimensional sound output section 26 increases the sound pressure when the azimuthal angle θ increases, and decreases the sound pressure when the azimuthal angle θ decreases. The three-dimensional sound output section 26 varies the sound pressure particularly when the frequency corresponding to the blood flow velocity f₀ is, for example, 800 Hz or more.

FIG. 13 shows one example of a method of controlling the sound pressure with which the speakers 29-1, 29-2, . . . , 29-n arranged on the right and left with respect to an azimuthal angle are driven. For example, the sound pressure of the speakers 29-1, 29-2, . . . , 29-n arranged on the left with respect to the operator 32 is increased, or the sound pressure of the speakers 29-1, 29-2, . . . , 29-n arranged on the left is decreased.

On the contrary, the sound pressure of the speakers 29-1, 29-2, . . . , 29-n arranged on the left with respect to the operator 32 is decreased, or the sound pressure of the speakers 29-1, 29-2, . . . , 29-n arranged on the left is increased. In addition, the difference of the sound pressure of the speakers 29-1, 29-2, . . . , 29-n arranged on the left needs to be, for example, 20 dB or more in the case of the sinusoidal wave.

Furthermore, the three-dimensional sound output section 26 changes the frequency characteristic with which at least one of the speakers 29-1, 29-2, . . . , 29-n is driven in accordance with the elevation angle φ. FIG. 14 shows one example of a method of controlling the sound pressure with which the speakers 29-1, 29-2, . . . , 29-n for frequencies using the elevation angle +as a parameter are driven. For example, when the elevation angle φ is, for example, 90° and great, the three-dimensional sound output section 26 decreases the sound pressure at a low frequency band, and then drives the speakers 29-1, 29-2, . . . , 29-n. Moreover, when the elevation angle φ is, for example, 90° and great, the three-dimensional sound output section 26 changes to a frequency characteristic which increases the sound pressure at a high frequency band, and then drives the speakers 29-1, 29-2, . . . , 29-n.

Furthermore, when the elevation angle φ is, for example, 0° and small, the three-dimensional sound output section 26 increases the sound pressure at a low frequency band, and changes to a frequency characteristic which decreases the sound pressure at a high frequency band, and then drives the speakers 29-1, 29-2, . . . , 29-n. In addition, the frequency is 1.4 KHz and sensitivity is high on the front side.

Next, in the second sound scheme, the sound pressure is varied when at least one of the speakers 29-1, 29-2, . . . , 29-n is driven by the three-dimensional sound output section 26 in accordance with the velocity f₀ of the specimen 4 such as blood flow. For example, the three-dimensional sound output section 26 increases the sound pressure when the blood flow velocity f₀ is high, and decreases the sound pressure when the blood flow velocity f₀ is low.

Furthermore, the three-dimensional sound output section 26 may change the reverberation amount of sound with which at least one of the speakers 29-1, 29-2, . . . , 29-n is driven in accordance with the velocity f₀ of the specimen 4 such as blood flow.

In connection with this, the three-dimensional sound output section 26 varies the phase difference with which the speakers 29-1, 29-2, . . . , 29-n arranged on the right and left with respect to the operator 32 are driven in accordance with the azimuthal angle θ. The phase difference of the speakers 29-1, 29-2, . . . , 29-n arranged on the right and left is, for example, 800 Hz or less.

A method of calculating the phase difference is as follows: The distance between the display 23 and the eye of the operator 32 is ra (e.g., 100 cm), the velocity of sound is C (=34000 cm/s), and the distance from the center of the head of the operator 32 to the ear is H (e.g., 12 cm). One example of the relation among a frequency f, a wavelength λ and Δra/λ is shown in the following table.

TABLE 1

| Frequency f | Wavelength λ | [symbol] r/λ |
|---|---|---|
| 100 Hz | 340 cm | 1/40 |
| 1 KHz | 34 cm | 1/4 |
| 10 KHz | 3.4 cm | 3 |

There is a limitation in the region where an azimuth can be separated by the phase difference:

−π<2π*Δra/λ<π.

The calculation of the phase difference in the two-dimensional plane is as shown below:

Δr=\sqrt{r²+H²+2*r*H*cos θ}−\sqrt{r²+H²−2*r*H*cos θ}

The three-dimensional sound output section 26 may use, for example, a sinusoidal signal or noise as the sound source.

Furthermore, when at least one of the speakers 29-1, 29-2, . . . , 29-n is driven in accordance with the elevation angle φ, the three-dimensional sound output section 26 changes to a frequency characteristic as shown in FIG. 14 to control the sound pressure with which the speaker 29-1, 29-2, . . . , 29-n are driven.

Next, in the third sound scheme, the frequency is varied when at least one of the speakers 29-1, 29-2, . . . , 29-n is driven by the three-dimensional sound output section 26 in accordance with the velocity f₀ (vector norm N) of the specimen 4 such as blood flow. The three-dimensional sound output section 26 increases the frequency when the blood flow velocity f₀is high, and decreases the frequency when the blood flow velocity f₀ is low.

In connection with this, the three-dimensional sound output section 26 varies the sound pressure with which at least one of the speakers 29-1, 29-2, . . . , 29-n is driven in accordance with the azimuthal angle θ. For example, the three-dimensional sound output section 26 controls the sound pressure with which the speakers 29-1, 29-2, . . . , 29-n arranged on the right and left with respect to the azimuthal angle 0 are driven, for example, as shown in FIG. 13.

Furthermore, the three-dimensional sound output section 26 varies the phase difference with which the speakers 29-1, 29-2, . . . , 29-n arranged on the right and left with respect to the operator 32 are driven in accordance with the azimuthal angle θ. The phase difference of the speakers 29-1, 29-2, . . . , 29-n arranged on the right and left is, for example, a frequency of 800 Hz or less. The method of calculating the phase difference is as described above.

Moreover, when at least one of the speakers 29-1, 29-2, . . . , 29-n is driven in accordance with the elevation angle φ, the three-dimensional sound output section 26 changes to the frequency characteristic as shown in FIG. 14 to control the sound pressure with which the speaker 29-1, 29-2, . . . , 29-n are driven.

The plurality of speakers 29-1 to 29-n are not exclusively arranged in the three-dimensional space surrounding the ultrasonic Doppler diagnostic apparatus main body 30, the operator 32, the bed 31, and the subject 33 on the bed 31. For example, it is possible to use a two-channel spatial sound system in which the speakers 16-1, 16-n are respectively arranged on the right and left with respect to the operator 32 such as the doctor, that is, a three-dimensional (3D) binaural system, a sound scheme using 7.1 channels, or a sound scheme using 5.1 channels.

The sound scheme using 7.1 channels uses speakers in front of the center, on the left, on the right, on the rear left, on the rear right, on both sides and for a woofer, with respect to the operator 32 such as the doctor. The sound scheme using 5.1 channels uses speakers in the center, on the left, on the right, on the rear left, on the rear right, and for a woofer.

In the case of using the right and left two-channel binaural system, for example, the two speakers 16-1, 16-n of the arrangement of speakers 16-1, 16-2, . . . , 16-n, the three-dimensional sound output section 26 adds a pseudo-characteristic using the transfer characteristic of spatial sound in accordance with the elevation angle φ, and then drives the two speakers 16-1, 16-n. In this case, the three-dimensional sound output section 26 changes at least the frequency characteristic as a pseudo-characteristic in accordance with the elevation angle φ, and then drives the two speakers 16-1, 16-n. Otherwise, the three-dimensional sound output section 26 adds reverberation, and then drives the two speakers 16-1, 16-n.

In addition, in the case of using the right and left two-channel binaural system, no speaker is disposed in the direction of the elevation angle φ. Thus, the three-dimensional sound output section 26 changes at least the frequency characteristic as a pseudo-characteristic in accordance with the elevation angle φ, and then drives the two speakers 16-1, 16-n. Otherwise, the three-dimensional sound output section 26 adds reverberation, and then drives the two speakers 16-1, 16-n.

In the case of using the right and left two-channel binaural system, the three-dimensional sound output section 26 provides a phase difference corresponding to the azimuthal angle θ and the elevation angle φ between the two speakers 16-1, 16-n, and then drives the speakers.

In the case of using the right and left two-channel binaural system, the three-dimensional sound output section 26 provides a gain difference between, for example, the two speakers 16-1, 16-n of the right and left two channels, and then drives the speakers. The three-dimensional sound output section 26 corrects high and low frequency characteristics on the basis of spatial sound data, and provides the corrected high and low frequency characteristics to, for example, the two speakers 16-1, 16-n, and then drives the speakers. This improves the ability to discriminate between the direction of the azimuthal angle θ and the direction of the elevation angle φ.

In the case of using the right and left two-channel binaural system, the three-dimensional sound output section 26 uses, as a sound source, noise which has been subjected to the amplitude modulation (AM) by the envelope of the waveform of the Doppler signal, such as the white noise. This makes it possible to improve the ability to separate the direction of the azimuthal angle θ from the direction of the elevation angle φ.

In the case of using the right and left two-channel binaural system, the three-dimensional sound output section 26 filters, as the sound source, the sinusoidal wave or white noise in accordance with the center frequency and dispersion of the waveform of the Doppler signal, and subjects the signal generated by the filtering to the amplitude modulation (AM) by the envelope of the waveform of the Doppler signal, and then uses the modulated signal. Thus, the three-dimensional sound output section 26 can output a sound in which the frequency of the Doppler signal is in proportion to the blood flow velocity.

The three-dimensional sound output section 26 changes, for example, the sound pressure or the frequency characteristic in accordance with the degree of turbulence and pulsation of the specimen 4 such as blood flow acquired by the three-dimensional information acquisition section 25, and then drives the two speakers 29-1, 29-n. The three-dimensional sound output section 26 varies, for example, the phase difference between the two speakers 16-1, 16-n, and then drives the speakers 29-1 to 29-n.

The colorization section 27 colorizes the blood flow directions in the three-dimensional ultrasonic image data for the range gate RG generated by the three-dimensional image data generation section 24, in accordance with the velocity f₀ (vector norm N), the azimuthal angle θ, the elevation angle φ, the degree of turbulence, the pulsation, etc., of the specimen 4 such as blood flow acquired by the three-dimensional information acquisition section 25. In this colorization, for example, blood flow moving toward the ultrasonic probe 1 is colored red, and blood flow moving away from the ultrasonic probe 1 is colored blue.

The indication section 28 indicates, on the display 23, the three-dimensional ultrasonic image data for the range gate RG generated by the three-dimensional image data generation section 24. The indication section 28 indicates, on the display 23, the three-dimensional ultrasonic image data for the range gate RG in which the blood flow directions have been colored by the colorization section 27.

Next, the operation of the apparatus having such a configuration is described.

A plurality of ultrasonic transducers of the ultrasonic probe 1 are, for example, electronically scanned by the scan wave transmitting/receiving section 20. When the ultrasonic transducers are sequentially driven, the ultrasonic probe 1 scans with the ultrasonic multibeam M. Thus, the ultrasonic multibeam M is sent to the range gate RG including the specimen 4 such as blood flowing in the blood vessel 3 within the living body 2 such as the human body.

The ultrasonic probe 1 receives a reflected wave from the region including the range gate RG, and outputs a signal from each of the ultrasonic transducers. The scan wave transmitting/receiving section 20 detects Doppler signals from the output signals of the ultrasonic transducers when the reflected wave from, for example, the range gate RG is received. The DSC 21 performs the digital conversion of the Doppler signals output from the scan wave transmitting/receiving section 20, and stores the digital Doppler signals in the storage section 22 such as an image memory. The DSC 21 reads the digital Doppler signals stored in the storage section 22 in accordance with the scanning of the display 23, and performs the analog conversion of the digital Doppler signals to display, on the display 23 in real time, an ultrasonic image of the range gate RG including the specimen 4 such as blood flowing in the blood vessel 3 within the living body 2 such as the human body.

That is, the three-dimensional image data generation section 24 of the DSC 21 performs the digital conversion of the Doppler signals output from the scan wave transmitting/receiving section 20, and stores, for example, digital Doppler signals for a preset scan period in the storage section 22, thereby acquiring a plurality of tomographic acquisition data (stack data). The three-dimensional image data generation section 24 reconstructs the plurality of tomographic acquisition data to generate three-dimensional ultrasonic image data (volume data) for the range gate RG within the living body 2 such as a human body.

The three-dimensional information acquisition section 25 acquires the three-dimensional fluid information for the specimen 4 such as blood flow within the range gate RG in the three-dimensional ultrasonic image data generated by the three-dimensional image data generation section 24, that is, the fluid vector data indicating the three-dimensional flow direction of the specimen 4 such as blood flow in the three-dimensional ultrasonic image data and the volume of the specimen 4 such as the blood flow. Specifically, the three-dimensional information acquisition section 25 acquires the velocity f₀, the azimuthal angle θ and the elevation angle φ of the specimen 4 such as blood flow, as the three-dimensional fluid information on the basis of the Doppler signals received by the ultrasonic transducers 6-1 to 6-4. Moreover, the three-dimensional information acquisition section 25 acquires the degree of turbulence and pulsation of the specimen 4 such as blood flow as the three-dimensional fluid information.

The three-dimensional sound output section 26 receives the velocity f₀ (vector norm N), the azimuthal angle θ and the elevation angle φ of the specimen 4 such as blood flow as the three-dimensional fluid information in the range gate RG acquired by the three-dimensional information acquisition section 25, and performs the sound conversion of the Doppler signals from the scan wave transmitting/receiving section 20 into Doppler sounds in a three-dimensional space in accordance with the blood flow velocity f₀ (vector norm N), the azimuthal angle θ and the elevation angle φ, and drives the plurality of speakers 29-1 to 29-n.

For example, as shown in FIG. 9, when the fluid vector data indicating the three-dimensional flow direction of the specimen 4 such as blood flow and the volume of the specimen 4 such as the blood flow is represented by the three-dimensional blood flow vector F₀ originating from the range gate RG, the three-dimensional sound output section 26 drives the plurality of speakers 29-1 to 29-n in accordance with the velocity f₀ (vector norm N), the azimuthal angle θ and the elevation angle φ of the specimen 4 such as blood flow indicated by the three-dimensional blood flow vector F₀.

In this case, for example, the three-dimensional sound output section 26 increases the sound pressure of the speakers 29-1 to 29-n arranged in the vector direction of the three-dimensional blood flow vector F₀, and decreases the sound pressure of the speakers 29-1 to 29-n as the distance in the three-dimensional direction from the vector direction of the three-dimensional blood flow vector F₀ increases. Thus, the operator 32 such as the doctor listens to the sound of at least one of the speakers 29-1, 29-2, . . . , 29-n, and thereby listens to the sound S corresponding to the velocity f₀, the azimuthal angle θ and the elevation angle φ of the specimen 4 such as blood flow indicated by the three-dimensional blood flow vector F₀ originating from the range gate RG.

Furthermore, the three-dimensional sound output section 26 can make variations by combining at least one or two of the sound pressure, phase difference and frequency characteristic of the three-dimensional Doppler sound with which at least one of the speakers 29-1, 29-2, . . . , 29-n is driven in accordance with the velocity f₀, the azimuthal angle θ and the elevation angle φ of the specimen 4 such as blood flow.

For example, in the first sound scheme, the three-dimensional sound output section 26 increases the frequency when the blood flow velocity f₀ is high, or decreases the frequency when the blood flow velocity f₀ is low, and then drives at least one of the speakers 29-1, 29-2, . . . , 29-n. In connection with this, as shown in, for example, FIG. 13, the three-dimensional sound output section 26 increases the sound pressure when the angle of elevation φ is great, or decreases the sound pressure when the angle of elevation φ is small, and then drives at least one of the speakers 29-1, 29-2, . . . , 29-n.

Furthermore, as shown in, for example, FIG. 14, when the angle of elevation φ is, for example, 90° and great, the three-dimensional sound output section 26 decreases the sound pressure at a low frequency band, and changes to a frequency characteristic which increases the sound pressure at a high frequency band, and then drives the speakers 29-1, 29-2, . . . , 29-n.

Moreover, when the angle of elevation φ is, for example, 0° and small, the three-dimensional sound output section 26 increases the sound pressure at a low frequency band, and changes to a frequency characteristic which decreases the sound pressure at a high frequency band, and then drives the speakers 29-1, 29-2, . . . , 29-n. Thus, the operator such as the doctor listens to a sound at a frequency corresponding to the blood flow velocity f₀, with a sound pressure corresponding to the azimuthal angle θ and with a frequency characteristic corresponding to the angle of elevation φ.

In the second sound scheme, for example, the three-dimensional sound output section 26 increases the sound pressure when the blood flow velocity f₀ is high, or decreases the sound pressure when the blood flow velocity f₀ is low, and then drives at least one of the speakers 29-1, 29-2, . . . , 29-n. The three-dimensional sound output section 26 may change the reverberation amount of sound with which at least one of the speakers 29-1, 29-2, . . . , 29-n is driven in accordance with the velocity f₀ of the specimen 4 such as blood flow.

In connection with this, the three-dimensional sound output section 26 varies the phase difference with which the speakers 29-1, 29-2, . . . , 29-n arranged on the right and left with respect to the operator 32 are driven in accordance with the azimuthal angle θ. The phase difference of the speakers 29-1, 29-2, . . . , 29-n arranged on the right and left is, for example, 800 Hz or less.

Moreover, the three-dimensional sound output section 26 changes the frequency characteristic in accordance with the angle of elevation φ as shown in FIG. 14 to control the sound pressure with which the speaker 29-1, 29-2, . . . , 29-n are driven. Thus, the operator such as the doctor listens to a sound with a sound pressure corresponding to the blood flow velocity f₀, with a phase difference corresponding to the azimuthal angle θ, and with a frequency characteristic corresponding to the angle of elevation φ.

In the third sound scheme, for example, the three-dimensional sound output section 26 increases the frequency when the blood flow velocity f₀ is high, or decreases the frequency when the blood flow velocity f₀ is low, and then drives at least one of the speakers 29-1, 29-2, . . . , 29-n. In connection with this, as shown in FIG. 13, the three-dimensional sound output section 26 controls the sound pressure with which the speakers 29-1, 29-2, . . , 29-n arranged on the right and left with respect to the azimuthal angle θ are driven.

Furthermore, the three-dimensional sound output section 26 varies the phase difference with which the speakers 29-1, 29-2, . . . , 29-n arranged on the right and left with respect to the operator 32 are driven in accordance with the azimuthal angle θ.

Moreover, the three-dimensional sound output section 26 changes the frequency characteristic in accordance with the angle of elevation φ as shown in FIG. 14 to control the sound pressure with which the speaker 29-1, 29-2, . . . , 29-n are driven. Thus, the operator such as the doctor listens to a sound at a frequency corresponding to the blood flow velocity f₀, with a sound pressure or a phase difference corresponding to the azimuthal angle θ, and with a frequency characteristic corresponding to the angle of elevation φ.

On the other hand, in the case of using the right and left two-channel binaural system, for example, the two speakers 16-1, 16-n, the three-dimensional sound output section 26 adds a pseudo-characteristic using the transfer characteristic of spatial sound in accordance with the angle of elevation φ, and then drives the two speakers 16-1, 16-n. In this case, the three-dimensional sound output section 26 changes at least the frequency characteristic as a pseudo-characteristic in accordance with the angle of elevation φ, or adds reverberation, and then drives the two speakers 16-1, 16-n.

In the case of using the right and left two-channel binaural system, the three-dimensional sound output section 26 provides a phase difference corresponding to the azimuthal angle θ and the angle of elevation φ between the two speakers 16-1, 16-n, and then drives the speakers. Moreover, in the case of using the right and left two-channel binaural system, the three-dimensional sound output section 26 provides a gain difference between, for example, the two speakers 16-1, 16-n of the right and left two channels, or corrects high and low frequency characteristics on the basis of spatial sound data and provides the corrected high and low frequency characteristics to the two speakers 16-1, 16-n, and then drives the speakers.

The three-dimensional sound output section 26 changes, for example, the sound pressure or the frequency characteristic in accordance with the degree of turbulence and pulsation of the specimen 4 such as blood flow acquired by the three-dimensional information acquisition section 25, and then drives the two speakers 29-1, 29-n. Otherwise, the three-dimensional sound output section 26 changes, for example, the phase difference between the two speakers 16-1, 16-n, and then drives the two speakers 29-1, 29-n.

The colorization section 27 colorizes the blood flow directions in the three-dimensional ultrasonic image data for the range gate RG generated by the three-dimensional image data generation section 24, in accordance with the three-dimensional fluid information such as the velocity f₀, the azimuthal angle θ, the angle of elevation φ, the degree of turbulence, the pulsation, etc., of the specimen 4 such as blood flow acquired by the three-dimensional information acquisition section 25. In this colorization, for example, blood flow moving toward the ultrasonic probe 1 is colored red, and blood flow moving away from the ultrasonic probe 1 is colored blue.

The indication section 28 indicates, on the display 23, the three-dimensional ultrasonic image data for the range gate RG including the specimen 4 such as blood flowing in the blood vessel 3 generated by the three-dimensional image data generation section 24. The indication section 28 indicates, on the display 23, the three-dimensional ultrasonic image data for the range gate RG in which the blood flow directions have been colored by the colorization section 27.

As described above, according to the one embodiment, the velocity f₀ (vector norm N), the azimuthal angle θ and the angle of elevation φ of the specimen 4 such as blood flow are acquired as the three-dimensional fluid information in the range gate RG on the basis of the Doppler signals detected by the ultrasonic transducers 6-1 to 6-4 in the ultrasonic probe 1. Variations are made by combining at least one or two of the sound pressure, phase difference and frequency characteristic of the three-dimensional Doppler sound when at least one of the speakers 29-1, 29-2, . . . , 29-n is driven in accordance with the blood flow velocity f₀ (vector norm N), the azimuthal angle θ and the angle of elevation φ.

Thus, the direction of blood flow can be acquired in a three-dimensional space in a coordinate system around the center of the ultrasonic probe 1 or around the range gate (RG) of a living body. For example, in the first sound scheme, the operator such as a doctor listens to a sound at a frequency corresponding to the blood flow velocity f₀, with a sound pressure corresponding to the azimuthal angle θ, and with a frequency characteristic corresponding to the angle of elevation φ, and can aurally know in the three-dimensional space the flow direction, volume, etc., of the specimen 4 which is a fluid such as blood flowing in the blood vessel 3.

Likewise, in the second sound scheme, the operator such as a doctor listens to a sound with a sound pressure corresponding to the blood flow velocity f₀, with a phase difference corresponding to the azimuthal angle θ, and with a frequency characteristic corresponding to the angle of elevation φ, and can aurally know in the three-dimensional space the flow direction, volume, etc., of the specimen 4 which is a fluid such as blood flowing in the blood vessel 3.

In the third sound scheme, the operator such as a doctor listens to a sound at a frequency corresponding to the blood flow velocity f₀, with a sound pressure or a phase difference corresponding to the azimuthal angle θ, and with a frequency characteristic corresponding to the angle of elevation φ, and can aurally know in the three-dimensional space the flow direction, volume, etc., of the specimen 4 which is a fluid such as blood flowing in the blood vessel 3.

In the case of using the right and left two-channel binaural system, the three-dimensional sound output section 26 can add a pseudo-characteristic using the transfer characteristic of spatial sound in accordance with the angle of elevation φ, and then drive the two speakers 16-1, 16-n. The three-dimensional sound output section 26 can provide a phase difference corresponding to the azimuthal angle θ and the angle of elevation φ between the two speakers 16-1, 16-n, and then drive the speakers.

Furthermore, in the case of using the right and left two-channel binaural system, the three-dimensional sound output section 26 can provide a gain difference between, for example, the two speakers 16-1, 16-n of the right and left two channels, and then drive the speakers. The three-dimensional sound output section 26 can correct high and low frequency characteristics on the basis of spatial sound data, and provide the corrected high and low frequency characteristics to, for example, the two speakers 16-1, 16-n, and then drive the speakers.

This also makes it possible to aurally know in the three-dimensional space the flow direction, volume, etc., of the specimen 4 which is a fluid such as blood flowing in the blood vessel 3.

It is to be noted that the present invention is not totally limited to the embodiment described above, and modifications of components can be made and embodied at the stage of carrying out the invention without departing from the spirit thereof. Moreover, suitable combinations of a plurality of components disclosed in the embodiment described above permit various inventions to be formed. For example, some of all the components shown in the embodiment described above may be eliminated. Moreover, components in different embodiments may be suitably combined together.

What is claimed is:

1. An ultrasonic diagnostic apparatus comprising:
a three-dimensional information acquisition section which acquires three-dimensional fluid information including at least a three-dimensional flow direction of a specimen on the basis of a Doppler signal output from an ultrasonic probe, the specimen being at least a fluid in a particular part; and
a three-dimensional sound output section which outputs the three-dimensional fluid information as a sound in a three-dimensional space.

2. The ultrasonic diagnostic apparatus according to claim 1, wherein
the three-dimensional information acquisition section acquires a velocity, an azimuthal angle and an angle of elevation of the specimen as the three-dimensional fluid information on the basis of fluid vector data indicating the three-dimensional flow direction and flow volume of the specimen found by the Doppler signal.

3. The ultrasonic diagnostic apparatus according to claim 2, wherein
the three-dimensional information acquisition section acquires the degree of turbulence and the pulsation of the specimen as the three-dimensional fluid information.

4. The ultrasonic diagnostic apparatus according to claim 1, wherein
the three-dimensional sound output section comprises a plurality of speakers, and drives at least one of the speakers corresponding to at least the three-dimensional flow direction of the specimen on the basis of the three-dimensional fluid information.

5. The ultrasonic diagnostic apparatus according to claim 2, wherein
the three-dimensional sound output section comprises a plurality of speakers, and drives at least one of the speakers on the basis of the velocity, the azimuthal angle and the angle of elevation of the specimen which have been acquired by the three-dimensional information acquisition section.

6. The ultrasonic diagnostic apparatus according to claim 5, wherein
the three-dimensional sound output section is configured to vary at least a sound pressure, a phase difference or a frequency characteristic of the three-dimensional sound with which at least one of the speakers is driven in accordance with the velocity, the azimuthal angle and the angle of elevation of the specimen.

7. The ultrasonic diagnostic apparatus according to claim 6, wherein
the three-dimensional sound output section changes at least one of the frequency characteristic, the sound pressure or a reverberation amount in accordance with the velocity of the specimen, and then drives at least one of the speakers.

8. The ultrasonic diagnostic apparatus according to claim 6, wherein
the three-dimensional sound output section changes at least one of the sound pressure and the phase difference in accordance with the azimuthal angle, and then drives at least one of the speakers.

9. The ultrasonic diagnostic apparatus according to claim 6, wherein
when the speakers are two-dimensionally arranged, the three-dimensional sound output section adds a pseudo-characteristic using the transfer characteristic of spatial sound in accordance with the angle of elevation, and then drives at least one of the speakers.

10. The ultrasonic diagnostic apparatus according to claim 9, wherein
the three-dimensional sound output section changes at least the frequency characteristic as the pseudo-characteristic in accordance with the angle of elevation, or adds reverberation, and then drives at least one of the speakers.

11. The ultrasonic diagnostic apparatus according to claim 6, wherein
in the case of using a binaural system in which the speakers are arranged as right and left two channels, the three-dimensional sound output section provides the phase difference corresponding to the azimuthal angle and the angle of elevation between the speakers.

12. The ultrasonic diagnostic apparatus according to claim 6, wherein
in the case of using a binaural system in which the speakers are arranged as right and left two channels, the three-dimensional sound output section provides a gain difference between the speakers of the right and left two channels, or corrects high and low frequency characteristics on the basis of spatial sound data and provides the corrected high and low frequency characteristics to the respective speakers.

13. The ultrasonic diagnostic apparatus according to claim 4, wherein
in the case of using a binaural system in which the speakers are arranged as right and left two channels, the three-dimensional sound output section uses, as a sound source, noise which has been subjected to amplitude modulation by the envelope of the waveform of the Doppler signal.

14. The ultrasonic diagnostic apparatus according to claim 4, wherein
in the case of using a binaural system in which the speakers are arranged as right and left two channels, the three-dimensional sound output section filters, as a sound source, a sinusoidal wave or white noise in accordance with the center frequency and dispersion of the waveform of the Doppler signal, and subjects the signal generated by the filtering to amplitude modulation by the envelope of the waveform of the Doppler signal, and then uses the modulated signal.

15. The ultrasonic diagnostic apparatus according to claim 3, wherein
the three-dimensional sound output section comprises a plurality of speakers arranged in a three-dimensional space, and drives the speakers in accordance with the degree of turbulence acquired by the three-dimensional information acquisition section.

16. The ultrasonic diagnostic apparatus according to claim 1, wherein
the ultrasonic probe sends an ultrasonic multibeam to the specimen, and receives a reflected wave from the specimen.

17. The ultrasonic diagnostic apparatus according to claim 4 or 5, wherein
the plurality of speakers are arranged in a two-dimensional or three-dimensional space.

18. The ultrasonic diagnostic apparatus according to claim 1, further comprising:
a display;
an indication section which indicates, on the display, three-dimensional ultrasonic image data generated on the basis of the Doppler signal output from the ultrasonic probe; and
a colorization section which colorizes the specimen in the three-dimensional ultrasonic image data in accordance with the three-dimensional fluid information including the three-dimensional flow direction.

19. A sound output method for an ultrasonic diagnostic apparatus comprising:
acquiring three-dimensional fluid information including at least a three-dimensional flow direction of a specimen on the basis of a Doppler signal output from an ultrasonic probe, the specimen being at least a fluid in a particular part; and
outputting the three-dimensional fluid information for the specimen as a three-dimensional sound.

20. The sound output method for the ultrasonic diagnostic apparatus according to claim 19, wherein
fluid vector data indicating the three-dimensional flow direction and flow volume of the specimen is obtained on the basis of the Doppler signal,
a velocity, an azimuthal angle and an angle of elevation of the specimen are acquired as the three-dimensional fluid information on the basis of the fluid vector data, and
at least one speaker is driven on the basis of the velocity, the azimuthal angle and the angle of elevation of the specimen.
